(19) United States
(12) Patent Application Publication
(10) Pub. No.: US 2023/0380770 A1
(43) Pub. Date: Nov. 30, 2023

(54) MULTI-CHANNEL ARRAY SENSOR FOR SPATIOTEMPORAL SIGNAL TRACKING

(71) Applicant: VENA VITALS, INC., Irvine, CA (US)

(72) Inventors: Ray Liu, Irvine, CA (US); Joshua Kim, Irvine, CA (US); Eugene Lee, Irvine, CA (US); Huy Eng Lim, Irvine, CA (US); Akhil Chaudhari, Irvine, CA (US)

(21) Appl. No.: 18/447,128

(22) Filed: Aug. 9, 2023

Related U.S. Application Data
(63) Continuation-in-part of application No. PCT/US22/15823, filed on Feb. 9, 2022.
(60) Provisional application No. 63/147,396, filed on Feb. 9, 2021.

(51) Int. Cl. A61B 5/00; G08C 17/02; G01D 3/028; A61B 5/021

(52) U.S. Cl. CPC A61B 5/7207; G08C 17/02; G01D 3/028; A61B 2562/04; A61B 5/6831; A61B 5/02125; A61B 5/002; A61B 5/6833

(57) ABSTRACT

Blood pressure measurement through the use of a sensor array system capable of tracking displacement, motion, environmental impact, and other electrical signals, and recalibration based on said tracking. The sensor array system may comprise a plurality of sensors, and each sensor may be capable of measuring one or more parameters. The system may further comprise an electronic board communicatively coupled to the sensor array. The electronic board may be capable of transmitting a plurality of parameter measurements from the sensor array to a computing device capable of detecting changes to the sensor array based on the plurality of parameter measurements. The changes to the sensor array may be detected by measuring an increased parameter reading from at least a first sensor and a decreased parameter reading from at least a second sensor compared to a baseline measurement.

### CROSS-REFERENCES TO RELATED APPLICATIONS

This application is a continuation-in-part and claims priority to PCT Application No. PCT/US2022/015823 filed Feb. 9, 2022, which claims priority to U.S. Provisional Application No. 63/147,396 filed Feb. 9, 2021, the specifications of which are incorporated herein in their entirety by reference.

### FIELD OF THE INVENTION

The present invention is directed to blood pressure measurement through the use of sensor arrays capable of tracking changes to a signal, and recalibration based on said tracking.

### BACKGROUND OF THE INVENTION

In the field of blood pressure monitoring, one or more parameters are measured by a sensor in order to derive a patient's blood pressure. When monitoring a parameter (such as capacitance, resistance, current, voltage, optical signals, radar, ultrasound, etc.) from an object (such as the radial artery), movement of that object relative to the reference in any direction (x, y, or z axes), or disturbances from mechanical, electromagnetic, temperature, physiological, environmental, or other sources, can impact the data being captured, causing inaccuracies. It is necessary to detect when this motion, displacement, or disturbance is occurring, by how much, and properly account for them in a monitoring parameter. Prior systems teach a second sensing parameter to directly measure the degree of disturbance, such as radar, ultrasound, optical, accelerometers, gyroscopes, or other sensing parameters that are different from the target sensing parameter. The use of a second sensing parameter requires the use of more energy and resources, causes prior systems to be more invasive overall, and may measure factors not affecting the blood pressure measurement. Thus, a present need exists for a blood pressure monitoring system capable of detecting displacement, motion, and other disturbance parameters by measuring the target sensing parameter alone.

### BRIEF SUMMARY OF THE INVENTION

It is an objective of the present invention to provide systems and methods that allow for detecting a change to a sensor by measuring a single sensing parameter, as specified in the independent claims. Embodiments of the invention are given in the dependent claims. Embodiments of the present invention can be freely combined with each other if they are not mutually exclusive.

The present invention features a system for tracking a measured physiological signal and utilizing spatiotemporal data to adjust the measured physiological signal for noise. In some embodiments, the system may comprise a sensor array. The sensor array may comprise a plurality of sensors, and each sensor may be capable of measuring one or more parameters. In some embodiments, each sensor of the plurality of sensors may comprise a pressure sensor (e.g. strain sensors), an optical sensor (e.g. infrared, visible light), an ultrasound sensor, a radar sensor, a spatiotemporal sensor, or a combination thereof. Each sensor may additionally be capable of measuring spatiotemporal properties. The one or more parameters measured by each sensor of the plurality of sensors may be an electrical or analog-to-digital signal selected from a group comprising a capacitance measurement, a resistance measurement, a current measurement, a voltage measurement, a radar measurement, an optical measurement, an ultrasound measurement, or a combination thereof. The system may further comprise an electronic board communicatively coupled to the sensor array. The electronic board may be capable of transmitting a plurality of parameter measurements from the sensor array to a computing device. The system may further comprise the computing device. The computing device may be capable of detecting a change to the sensor array based on the plurality of parameter measurements. The change to the sensor array may be detected by measuring an increased parameter reading from at least a first sensor of the plurality of sensors and a decreased parameter reading from at least a second sensor of the plurality of sensors compared to a baseline measurement. The baseline measurement may be established by the computing device based on an initial plurality of parameter measurements received by the electronics board.

The present invention features a method for tracking a measured physiological signal and utilizing spatiotemporal data to adjust the measured physiological signal for noise. In some embodiments, the method may comprise a sensor array comprising a plurality of sensors measuring a first and a second plurality of parameter measurements. Each sensor of the plurality of sensors may be capable of measuring one or more parameters. In some embodiments, each sensor of the plurality of sensors may comprise a pressure sensor, a spatiotemporal sensor, or a combination thereof. The single parameter measured by each sensor of the plurality of sensors may be an electrical or analog-to-digital signal selected from a group comprising a capacitance measurement, a resistance measurement, a current measurement, a voltage measurement, a radar measurement, an optical measurement, an ultrasound measurement, or a combination thereof. The method may further comprise an electronic board communicatively coupled to the sensor array transmitting the first and the second plurality of parameter measurements to a computing device. The method may further comprise the computing device establishing a baseline measurement based on the first plurality of parameter measurements. The method may further comprise the computing device detecting a change to the sensor array based on the second plurality of parameter measurements. The method may further comprise the computing device adjusting the baseline measurement based on the change to the sensor array. The change to the sensor array may be detected by measuring an increased parameter reading from at least a first sensor of the plurality of sensors and a decreased parameter reading from at least a second sensor of the plurality of sensors compared to a baseline measurement.

One of the unique and inventive technical features of the present invention is the adjustment of noise from a plurality of sensors through the use of spatiotemporal data. Without wishing to limit the invention to any theory or mechanism, it is believed that the technical feature of the present invention advantageously provides for more accurate measurement of the changes to the plurality of sensors due the exclusion of a wide variety of noise types. None of the presently known prior references or work has the unique inventive technical feature of the present invention.

For example, prior systems for adjusting signals from sensors for noise teach a method of redundant sensing. In the redundant sensing method, there are two or more similar sensors where one is measuring the signal and noise, while the other sensors are measuring just noise. It is possible in some applications where the noise signal can be subtracted out from the primary signal resulting in the primary signal. However, there are situations where the noise can have different magnitudes measured across all sensors including the primary sensor. In this case, the method for redundant sensing is inefficient.

Furthermore, the inventive technical feature of the present invention is counterintuitive. The reason that it is counterintuitive is because the inventive technical feature contributed to a surprising result. One skilled in the art would determine that if the noise varies in magnitude (including situations where one signal goes up and one goes down due to noise) across the sensors that it would be too difficult to utilize spatiotemporal information to filter out noise impacting the target signal for it to be worth implementing. Surprisingly, the present invention is able to implement a spatiotemporal mesh that is able to identify noise. even in large quantities or with variable magnitude, and filter it from the target signal to provide a more accurate final signal than prior systems. Thus, the inventive technical feature of the present invention contributed to a surprising result and is counterintuitive.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of a system of the present invention for identifying and tracking displacement of an object relative to a reference point.

FIG. 2 shows a flow chart of a method of the present invention for identifying and tracking displacement of an object relative to a reference point.

FIG. 3A shows an example of displacement of an object relative to a sensor. FIG. 3B shows an example of displacement of an object relative to a plurality of sensors. FIG. 3C shows an alternate example of displacement of an object relative to a plurality of sensors.

FIG. 4 shows a photograph of a multi-channel sensor array that can be implemented in the system of the present invention.

FIG. 5 shows a plurality of embodiments of the sensor of the present invention. Within each sensor are two sets of two multiplexed sensors for a total of four sensing elements.

FIG. 6 shows a plurality of alternative embodiments of the sensor of the present invention, each embodiment comprising a multiplexor.

FIG. 7 shows a schematic of an adaptive filter that may be implemented in the system of the present invention for filtering noise, creep, hysteresis, motion, temperature, electromagnetic signals, intrinsic sensor noise (e.g. sensor drift), or a combination thereof out of the signals provided by the sensor array.

FIG. 8A shows a strain sensor that may be implemented in the system of the present invention measuring a radial artery in a diastolic state (in between beats). FIG. 8B shows the strain sensor that may be implemented in the system of the present invention measuring a radial artery in a systolic state (blood being pumped).

FIG. 9A shows a photograph of the components of the system of the present invention. FIG. 9B shows a photograph of a prototype of the system of the present invention in use on a patient.

FIGS. 10A-10D show a graph of a plurality of signals gathered from the sensor array of the present invention, both individually and combined into a 3-dimensional spatiotemporal graph showing the measured signal and the spatial position of each individual sensor.

FIG. 11A shows a graph of an arterial line signal over time after baseline correction using a multichannel sensor. FIG. 11B shows the same graph as FIG. 11A with the original, non-corrected signal and a line showing the systolic/diastolic blood pressure of the corrected signal.

FIG. 12 shows an example graph of measuring pulse transit time across multiple sensors in order to track arterial activity over a certain area covered by the plurality of sensors.

FIG. 13 shows an example graph of pulse wave analysis executed on an arterial signal gathered by a sensor of the present invention.

### DETAILED DESCRIPTION OF THE INVENTION

Following is a list of elements corresponding to a particular element referred to herein:
100 detection system
200 sensor array
201 sensor
300 electronic board
301 first communication component
302 first processor
303 first memory component
400 computing device
401 second communication component
402 second processor
403 second memory component

The present invention applies an array of original single-parameter sensors to serve as an array of reference points that allows the present invention to indirectly measure the degree of displacement or motion without having to use a different sensing parameter to directly measure it. The present invention is additionally able to detect temperature, electromagnetism, or any other environmental parameter, analyze their impact on the signals gathered from the plurality of parameter measurements, and subtract this noise from the parameter measurements to allow for a more accurate final product. This technique allows for a system to simultaneously track the signal changes from each point on the array to determine if the object has moved or been displaced. An algorithm-based approach is then used to translate the detected signal changes across the array to deduce the direction and magnitude of the displacement and differentiate displacement-caused signal changes from real physiological-caused signal changes. The present invention uses an array of sensors that independently do not measure motion/displacement and apply them in a way to measure motion/displacement without the need to integrate a different type of sensor (such as optical sensors, accelerometers, or gyroscopes). The entire array of sensors can be used to deduce whether displacement is occurring, triangulate between the different sensors the actual magnitude of the displacement, and reset or re-calibrate the signal accordingly. The sensor array may comprise at least two individual sensors. In some embodiments, a sensors array comprises 6 to 10 sensors. A size of each sensor may be 2 mm to 3 mm by 2 mm to 3 mm.

The present invention features a system (100) for tracking a measured

physiological signal and utilizing spatiotemporal data to adjust the measured physiological signal for noise. In some embodiments, the system (100) may comprise a sensor array (200). The sensor array (200) may comprise a plurality of sensors, and each sensor (201) may be capable of measuring one or more parameters. In some embodiments, each sensor of the plurality of sensors may comprise a pressure sensor capable of measuring capacitance, resistance, current, and/or voltage, a spatiotemporal sensor capable of measuring optical, radar, and/or ultrasound signals, or a combination thereof. The single parameter measured by each sensor of the plurality of sensors may be an electrical or analog-to-digital signal selected from a group comprising a capacitance measurement, a resistance measurement, a current measurement, a voltage measurement, a radar measurement, an optical measurement, an ultrasound measurement, or a combination thereof. The system (100) may further comprise an electronic board (300) communicatively coupled to the sensor array (200). The electronic board (300) may be capable of transmitting a plurality of parameter measurements from the sensor array (200) to a computing device (400). In some embodiments, the electronic board (300) may transmit the plurality of parameter measurements to the computing device (400) through low-energy Bluetooth transmissions. The system (100) may further comprise the computing device (400). The computing device (400) may be capable of detecting changes to the sensor array (200) based on the plurality of parameter measurements. The changes to the sensor array (200) may comprise movement (e.g. displacement), environmental features (e.g. temperature), or a combination thereof.

Changes to the sensor array (200) may be detected by measuring an increased parameter reading or a decreased parameter reading from one or more sensors of the plurality of sensors compared to the baseline measurement. The baseline measurement may be established by the computing device (400) based on an initial plurality of parameter measurements received by the electronics board (300). The computing device (400) may be capable of deriving a spatiotemporal data set from the plurality of parameter measurements, detecting noise in the spatiotemporal data set, and adjusting, based on the plurality of parameter measurements, the baseline measurement with respect to the noise. In some embodiments, the computing device (400) may be further capable of converting a measurement into a blood pressure measurement. In some embodiments, the measurement may comprise an electrical or analog-to-digital signal selected from a group comprising a capacitance measurement, a resistance measurement, a current measurement, a voltage measurement, a radar measurement, an optical measurement, an ultrasound measurement, or a combination thereof. In some embodiments, the system (100) may further comprise an attachment component connected to the sensor array (200) for attaching the sensor array (200) to an external surface. The attachment component may be selected from a group comprising a strap and an adhesive. The external surface may be a portion of skin covering a carotid artery, a radial artery, or any other artery near the surface of the skin or another layer such as or including surgical dressing disposed on a portion of skin of the patient. In some embodiments, the electronic board (300) may further comprise an adaptive filter for filtering noise, creep, hysteresis, motion, temperature, electromagnetic signals, intrinsic sensor noise (e.g. sensor drift), or a combination thereof from the plurality of parameter measurements (see FIG. 8). In some embodiments, each sensor may additionally experience intrinsic noise, meaning each individual sensor itself can drift. Having multichannel sensors can help us discern this intrinsic noise from the target signal. In some embodiments, a sensor array may be capable of filtering intrinsic noise of individual sensors by detecting a common signal change in all sensors as a result of stretching or compression of the individual sensors and subtracting the noise caused by the common signal change from the plurality of signals received from the sensor array. The common signal change may be the result of displacement. The sensor array may additionally be capable of filtering out temperature impact, electromagnetic noise, or any other change that affects all sensors of the sensor array in some way (increased or decreased parameter reading) in a similar manner. In some embodiments, the noise may comprise noise or disturbances picked up by one or more sensors.

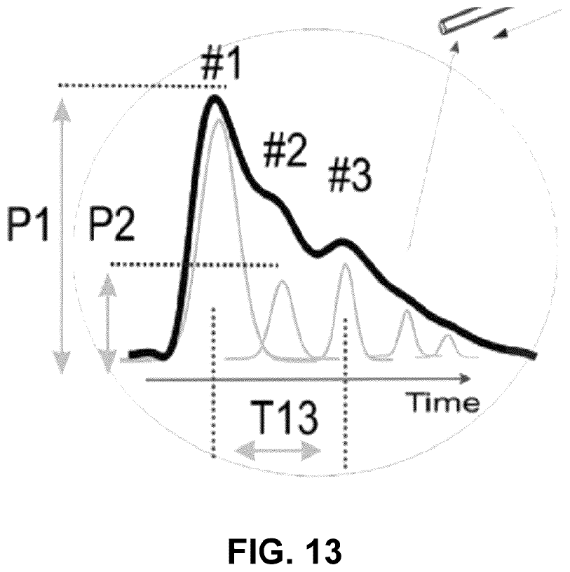

In some embodiments, the computing device (400) may be further capable of measuring pulse transit time (PTT) between a first sensor of the plurality of sensors and a second sensor of the plurality of sensors. An example of this can be seen in FIG. 13. PTT provides a basis for ubiquitous blood pressure monitoring. PTT is the time delay for the pressure wave to travel between two arterial sites and can be estimated simply from the relative timing between proximal and distal arterial waveforms. PTT is often inversely related to BP. The computing device (400) may be further capable of analyzing a pulse wave gathered by one or more sensors of the plurality of sensors. Pulse wave analysis is a technique to extract specific features within one pulse waveform/cardiac cycle. Traditionally pulse wave is applied to 1-D signals, usually from optical measurements (PPG). However, the present invention is able to perform a 3D pulse wave analysis that can potentially be more accurate than 1-D signals. This is due to the spatial information gathered by the plurality of sensors of the present invention, contrary to prior systems that do not gather spatial information and therefore only generate 1-D signals.

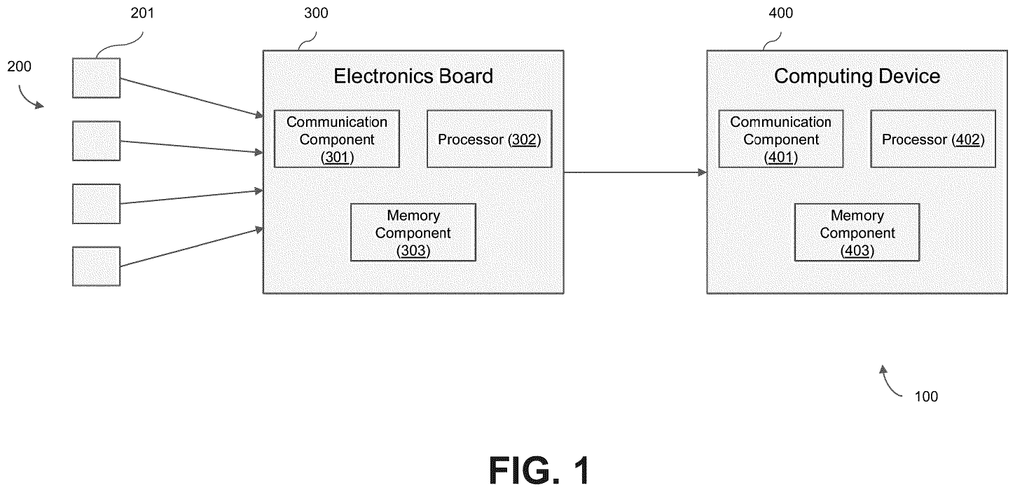

Referring now to FIG. 1, the present invention features a system (100) for tracking a measured physiological signal and utilizing spatiotemporal data to adjust the measured physiological signal for noise. In some embodiments, the system (100) may comprise a sensor array (200) comprising a plurality of sensors. Each sensor (201) may be capable of measuring one or more parameters. In some embodiments, each sensor of the plurality of sensors may comprise a pressure sensor capable of measuring capacitance, resistance, current, and/or voltage, a spatiotemporal sensor capable of measuring optical, radar, and/or ultrasound signals, or a combination thereof. The single parameter measured by each sensor of the plurality of sensors may be an electrical or analog-to-digital signal selected from a group comprising a capacitance measurement, a resistance measurement, a current measurement, a voltage measurement, a radar measurement, an optical measurement, an ultrasound measurement, or a combination thereof. The system (100) may further comprise an electronic board (300) communicatively coupled to the sensor array (200). In some embodiments, the electronic board (300) may comprise a first communication component (301), a first processor (302) capable of executing computer-readable instructions, and a first memory component (303) comprising computer-readable instructions. The computer-readable instructions may comprise receiving a plurality of parameter measurements from the sensor array (200), and transmitting, by the first communication component (301), the plurality of parameter measurements. In some embodiments, the electronic board (300) may transmit the plurality of parameter measurements to the computing device (400) through low-energy Bluetooth transmissions. The system (100) may further comprise a computing device (400) communicatively coupled to the electronic board (300). The computing device (400) may comprise a second communication component (401), a second processor (402) capable of executing computer-readable instructions, and a second memory component (403) comprising computer-readable instructions. The computer-readable instructions may comprise receiving, by the second communication component (401), a first plurality of parameter measurements and a second plurality of parameter measurements from the electronic board (300). The computer-readable instructions may further comprise establishing, based on the first plurality of parameter measurements, a baseline measurement. The computer-readable instructions may further comprise deriving a spatiotemporal data set from the plurality of parameter measurements, detecting noise in the spatiotemporal data set, and adjusting, based on the plurality of parameter measurements, the baseline measurement with respect to the noise. In some embodiments, the spatiotemporal data set comprises a spatiotemporal mesh. The spatiotemporal mesh may be generated by accepting the plurality of parameter measurements from the sensor array and utlizing interpolation to estimate the data between the individual sensors of the sensor array. The interpolated data may be used to identify patterns in the parameter measurements and adjust the sensor readings accordingly to fall in line with these patterns, thus accounting for noise.

Changes to the sensor array (200) may be detected by measuring an increased parameter reading or a decreased parameter reading from one or more sensors of the plurality of sensors compared to the baseline measurement. In some embodiments, the second memory component (403) may further comprise instructions for converting the measurement into a blood pressure measurement. In some embodiments, the measurement may comprise an electrical or analog-to-digital signal selected from a group comprising a capacitance measurement, a resistance measurement, a current measurement, a voltage measurement, a radar measurement, an optical measurement, an ultrasound measurement, or a combination thereof. In some embodiments, the system (100) may further comprise an attachment component connected to the sensor array (200) for attaching the sensor array (200) to an external surface. The attachment component is selected from a group comprising a strap and an adhesive. The external surface may be a portion of skin covering a carotid artery, a radial artery, or any other artery near the surface of the skin or another layer such as or including surgical dressing disposed on a portion of skin of the patient. In some embodiments, the electronic board (300) may further comprise an adaptive filter for filtering noise, creep, hysteresis, motion, temperature, electromagnetic signals, intrinsic sensor noise (e.g. sensor drift), or a combination thereof from the plurality of parameter measurements (see FIG. 8).

In some embodiments, the first communication component (301) may comprise a wired connection between the sensor array (200) and the electronic board (300), a wireless connection between the sensor array (200) and the electronic board (300) such that the sensor array (200) comprises a wireless transmitter and the electronic board (300) comprises a wireless receiver. The wireless connection may comprise Bluetooth, LoRa, radiofrequency, or any other kind of wireless communication type. In some embodiments, the second communication component (401) may comprise a wired connection between the electronic board (300) and the computing device (400), a wireless connection between the electronic board (300) and the computing device (400) such that the electronic board (300) comprises a wireless transmitter and the computing device (400) comprises a wireless receiver. The wireless connection may comprise Bluetooth, LoRa, radiofrequency, or any other kind of wireless communication type.

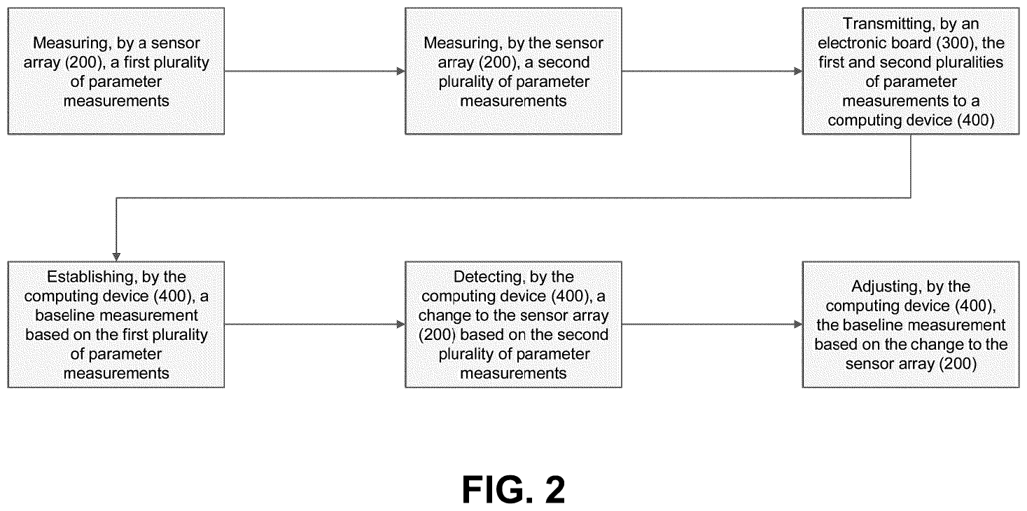

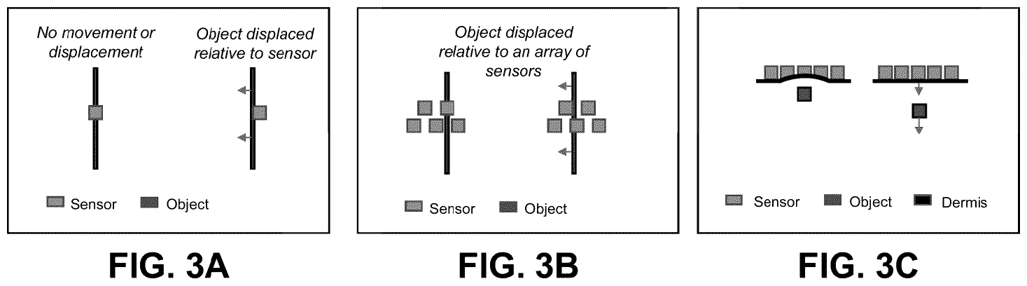

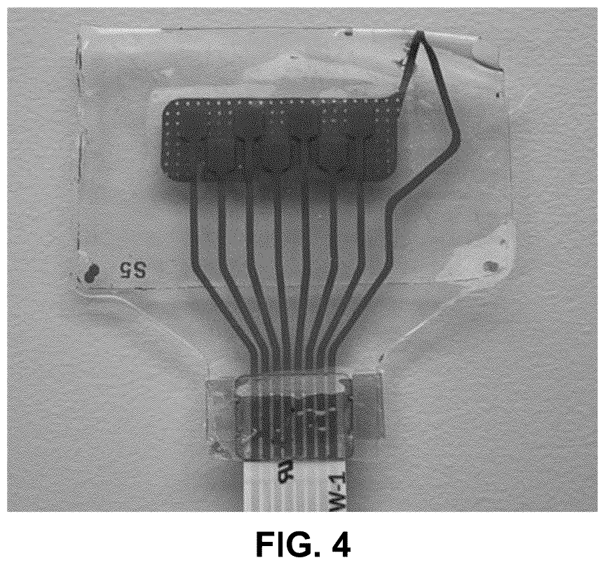

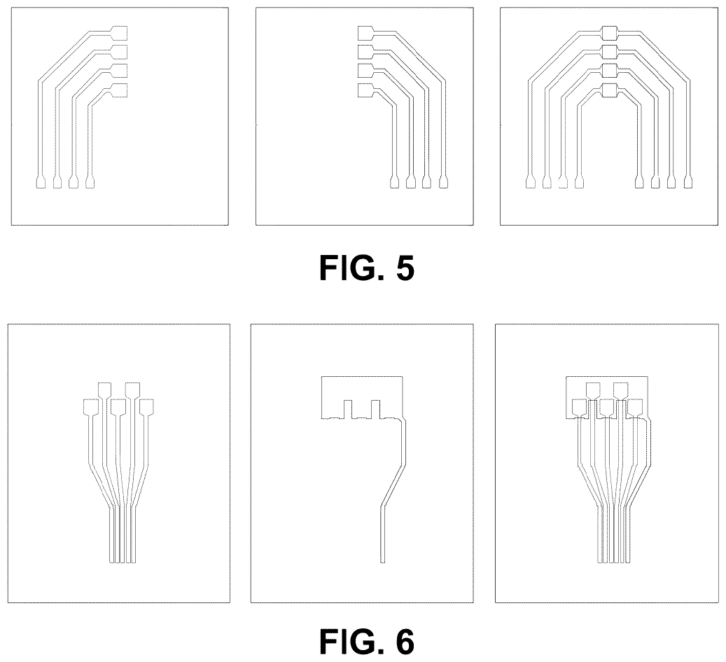

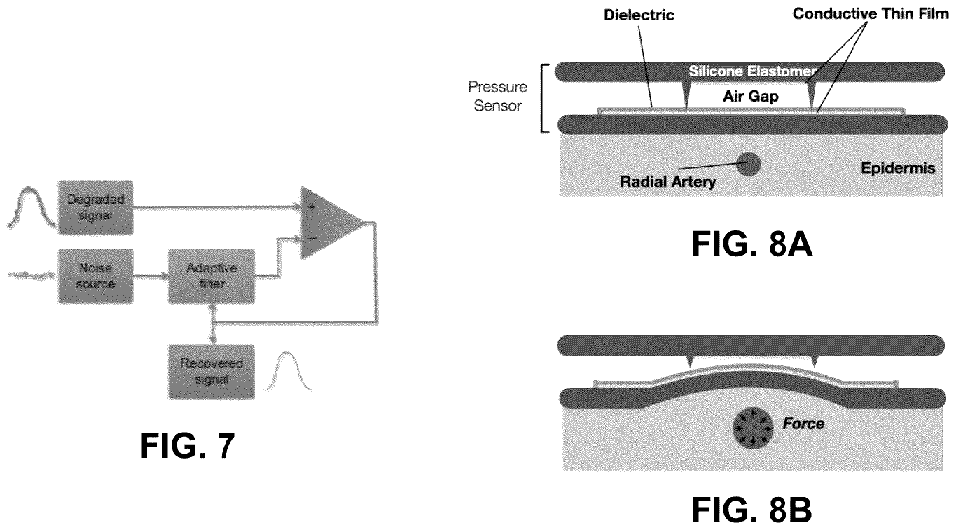

Referring now to FIG. 2, the present invention features a method for tracking a measured physiological signal and utilizing spatiotemporal data to adjust the measured physiological signal for noise. In some embodiments, the method may comprise a sensor array (200) comprising a plurality of sensors measuring a first plurality of parameter measurements. Each sensor (201) of the plurality of sensors may be capable of measuring one or more parameters. In some embodiments, each sensor of the plurality of sensors may comprise a pressure sensor capable of measuring capacitance, resistance, current, and/or voltage, a spatiotemporal sensor capable of measuring optical, radar, and/or ultrasound signals, or a combination thereof. The single parameter measured by each sensor of the plurality of sensors may be an electrical or analog-to-digital signal selected from a group comprising a capacitance measurement, a resistance measurement, a current measurement, a voltage measurement, a radar measurement, an optical measurement, an ultrasound measurement, or a combination thereof. The method may further comprise measuring, by the sensor array (200), a second plurality of parameter measurements. The method may further comprise an electronic board (300) communicatively coupled to the sensor array (200) transmitting the first plurality of parameter measurements and the second plurality of parameter measurements to a computing device (400). The method may further comprise the computing device (400) establishing a baseline measurement based on the first plurality of parameter measurements. The method may further comprise deriving, by the computing device (400), a spatiotemporal data set from the plurality of parameter measurements, detecting noise in the spatiotemporal data set, and adjusting, based on the plurality of parameter measurements, the baseline measurement with respect to the noise.

Changes to the sensor array (200) may be detected by measuring an increased parameter reading or a decreased parameter reading from one or more sensors of the plurality of sensors compared to the baseline measurement. In some embodiments, the method may further comprise the computing device (400) converting a measurement into a blood pressure measurement. In some embodiments, the measurement may comprise an electrical or analog-to-digital signal selected from a group comprising a capacitance measurement, a resistance measurement, a current measurement, a voltage measurement, a radar measurement, an optical measurement, an ultrasound measurement, or a combination thereof. In some embodiments, may further comprise attaching, by an attachment component connected to the sensor array (200), the sensor array (200) to an external surface. The attachment component may be selected from a group comprising a strap and an adhesive. The external surface may be a portion of skin covering a carotid artery, a radial artery, or any other artery near the surface of the skin or another layer such as or including surgical dressing disposed on a portion of skin of the patient. In some embodiments, the method may further comprise an adaptive filter filtering noise, creep, hysteresis, motion, temperature, electromagnetic signals, intrinsic sensor noise (e.g. sensor drift), or a combination thereof from the plurality of parameter measurements (see FIG. 8).

The present invention features a system (100) for tracking a measured physiological signal and utilizing spatiotemporal data to adjust the measured physiological signal for noise. In some embodiments, the system (100) may comprise a sensor array (200) comprising a plurality of sensors. Each sensor (201) may be capable of measuring one or more parameters. The system may further comprise an electronic board (300) communicatively coupled to the sensor array (200). In some embodiments, the electronic board (300) may comprise a first communication component (301), a first processor (302) capable of executing computer-readable instructions, and a first memory component (303) comprising computer-readable instructions. In some embodiments, the computer-readable instructions may comprise receiving, from the sensor array (200), a plurality of parameter measurements, and transmitting, by the first communication component (301), the plurality of parameter measurements. The system may further comprise a computing device (400) communicatively coupled to the electronic board (300). In some embodiments, the computing device (400) may comprise a second communication component (401), a second processor (402) capable of executing computer-readable instructions, and a second memory component (403) comprising computer-readable instructions. In some embodiments, the computer-readable instructions may comprise receiving, by the second communication component (401), a first plurality of parameter measurements and a second plurality of parameter measurements from the electronic board (300), establishing, based on the first plurality of parameter measurements, a baseline measurement, detecting, based on the second plurality of parameter measurements, a change to the sensor array (200), and adjusting, based on the change to the sensor array (200), the baseline measurement. The change to the sensor array (200) may be detected by measuring an increased parameter reading from at least a first sensor of the plurality of sensors and a decreased parameter reading from at least a second sensor of the plurality of sensors compared to the baseline measurement. The computer-readable instructions may further comprise measuring pulse transit time between a first sensor of the plurality of sensors and a second sensor of the plurality of sensors, and analyzing a pulse wave gathered by one or more sensors of the plurality of sensors. Analyzing the pulse wave gathered by one or more sensors of the plurality of sensors further comprises extracting amplitude, phase, frequency, systolic blood pressure, diastolic blood pressure, dicrotic notch, a time difference between systolic and diastolic peaks, a rate of blood pressure change between systolic and diastolic peaks, a minimum blood pressure change between systolic and diastolic peaks, heart rate, heart rate variability, or a combination thereof from the pulse wave.

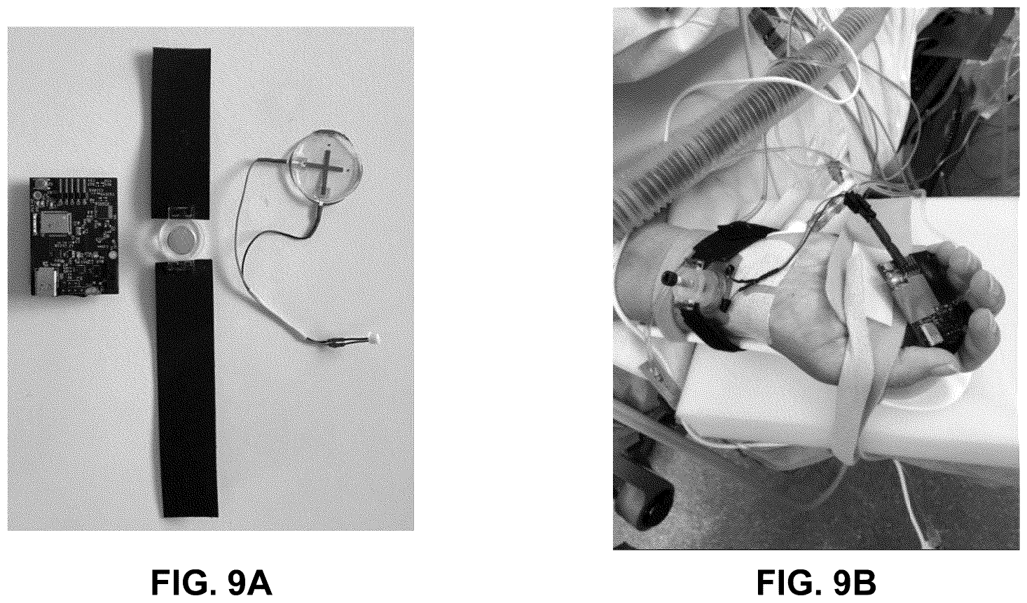

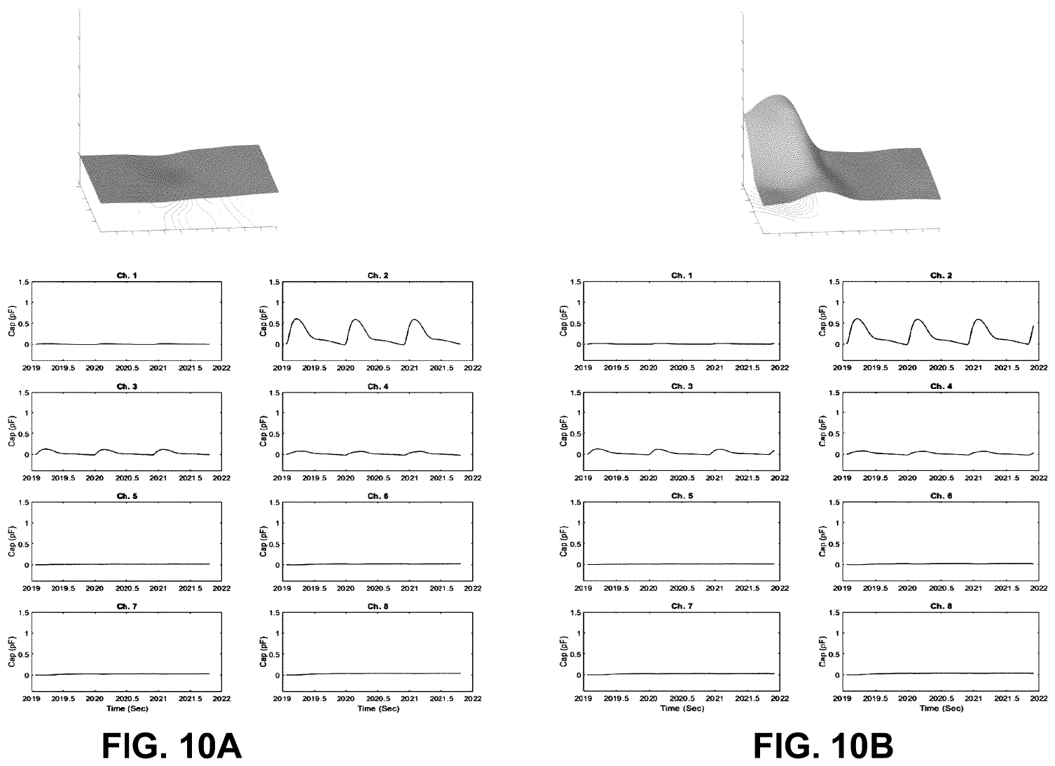

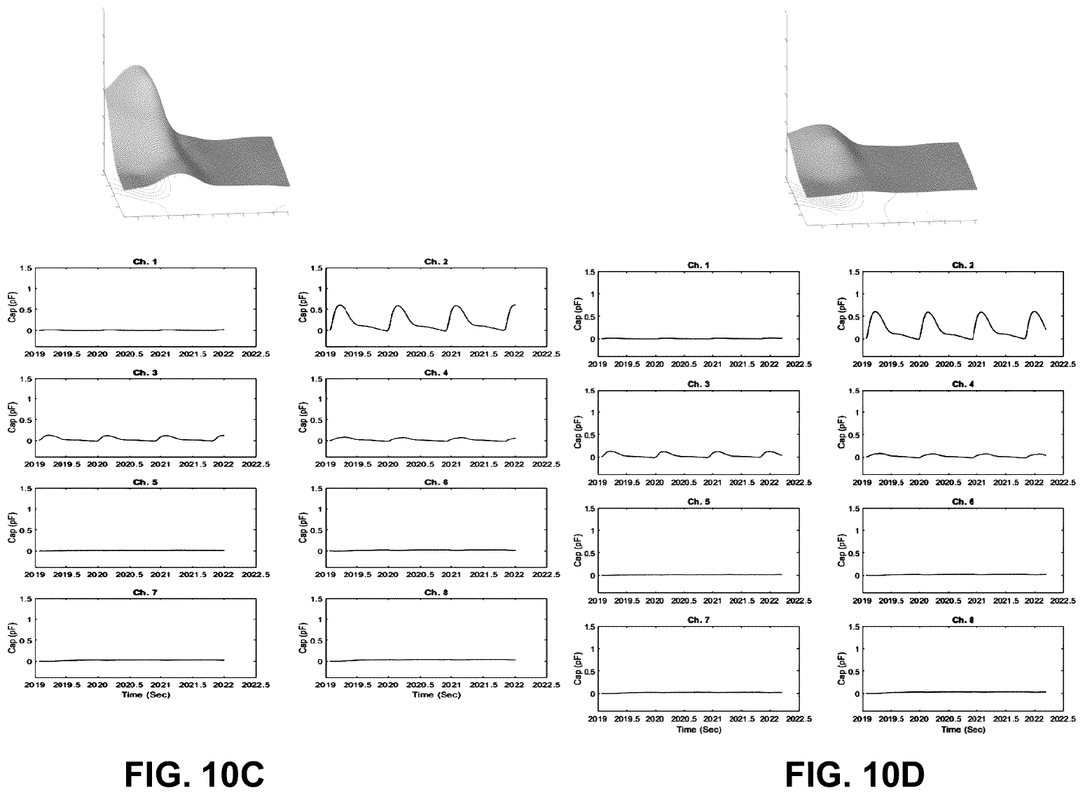

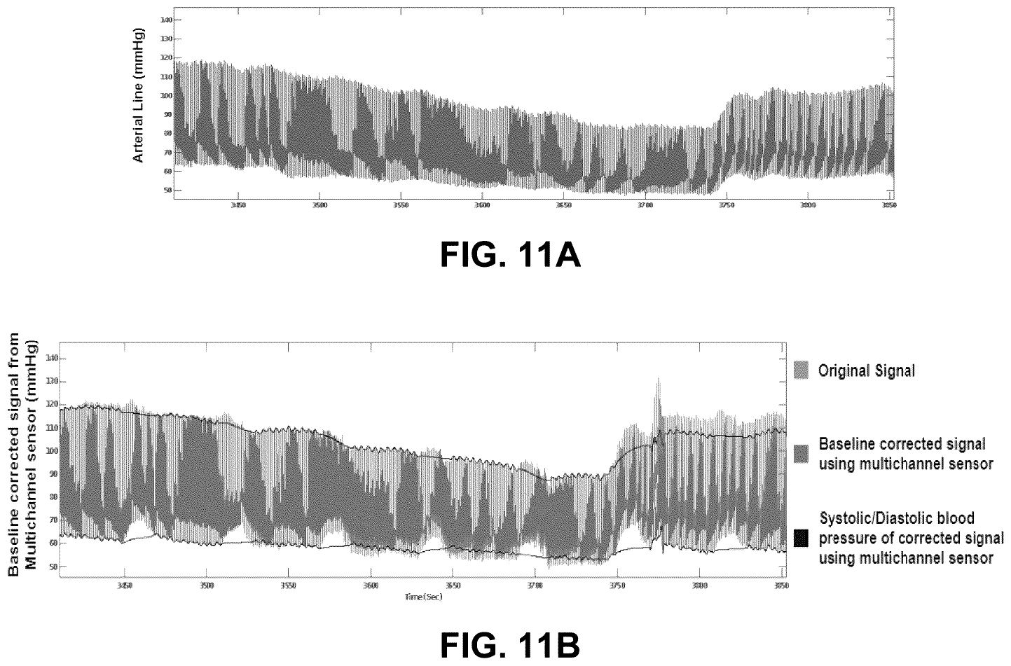

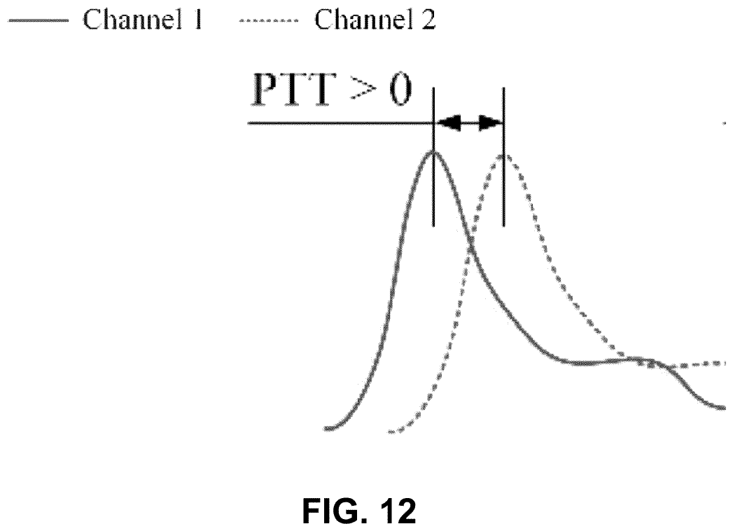

Referring now to FIG. 9A, a sensor of the sensor array may comprise a pressure sensor. The pressure sensor may comprise an attachment component allowing the pressure sensor to attach to a surface (e.g. skin above an artery of a patient). The attachment component may comprise an adhesive, a strap, or any other component allowing the sensor to be stabilized in place in contact with the surface. The pressure sensor may further comprise a first sensor component disposed on top of the attachment component, the first sensor component comprising a first polymer layer, a first conductive thin film layer disposed on top of the first polymer layer, and a dielectric layer disposed on top of the first conductive thin film layer. The first polymer layer may comprise a silicone elastomer. The first conductive thin film layer may comprise gold, platinum, copper, or any other conductive material. Definition of dielectric describes any material that can produce an electric field without having to conduct electricity. This means, the dielectric layer is a passivating and insulating layer to prevent conduction between two electrodes. Some examples include air, paralyne, silicone, ceramics (i.e., barium titanate, lead zirconate titanate), polyvinylidene fluoride (PVDF), and even composites such as silver nanoparticles embedded in silicone (as long as the silver particles do not overcome the percolation threshold to conduct electricity). The pressure sensor may further comprise a second sensor component connected to the first sensor component by one or more elastic ridges such that an air gap exists between the first sensor component and the second sensor component. The one or more elastic ridges may be moved in response to pressure beneath the first sensor component. In some embodiments, as seen in FIG. 9B, the first sensor component may additionally be moved in response to pressure beneath the first sensor component. The second sensor component may comprise a second conductive thin film layer and a second polymer layer disposed on top of the second conductive thin film layer. The second polymer layer may comprise a silicone elastomer. The second conductive thin film layer may comprise gold, platinum, copper, or any other conductive material.

A sensor of the sensor array may comprise a pressure sensor, an electromagnetic sensor (e.g an optical sensor, an ultrasound sensor, a radar sensor), a capacitive sensor, a resistive sensor, or a combination thereof. Each sensor may additionally comprise a thermometer, an accelerometer, a gyroscope, a magnetometer, a bioimpedance sensor, or a combination thereof, which may be auxiliary to the primary function of each sensor. Note that the presently claimed invention may be capable of measuring displacement and detecting disturbances affecting the displacement reading. Non-limiting examples of displacement measured by the present invention include movement of an artery relative to the array of sensors, such as the movement of a pulse throughout the body or wave propagation data detected by measuring when the artery expands in one area and pulls down in another. Non-limiting examples of disturbances detected by the present invention include environmental noise that affects one or more sensors similarly (e.g. temperature, electromagnetism), noise caused by surface topography (e.g. each sensor of the sensor array being placed on different inclines), and gradient noise that moves across one or more sensors. The present invention is capable of detecting these disturbances in the signals received from the sensor array and use spatiotemporal data to filter them from the plurality of sensors, thus resulting in a more accurate final product with less noise than that achieved by prior systems.

Instructions that cause at least one processing circuit to perform one or more operations are “computer-readable.” Physical storage media (memory components) includes RAM and other volatile types of memory; ROM, EEPROM, and other non-volatile types of memory; CD-ROM, CD-RW, DVD-ROM, DVD-RW, and other optical disk storage; magnetic disk storage or other magnetic storage devices; and any other tangible medium that can store computer-executable instructions that can be accessed and processed by at least one processing circuit. Transmission media can include signals carrying computer-executable instructions over a network to be received by a general-purpose or special-purpose computer.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for tracking a measured signal and utilizing spatiotemporal data to adjust the measured signal, the system (100) comprising:
a. a sensor array (200) comprising a plurality of sensors, each sensor (201) capable of measuring one or more parameters;
b. an electronic board (300) communicatively coupled to the sensor array (200) for transmitting a plurality of parameter measurements from the sensor array (200) to a computing device (400); and
c. the computing device (400) communicatively coupled to the electronic board (300) for detecting a change to the sensor array (200) based on the plurality of parameter measurements;
wherein the change to the sensor array (200) is detected by measuring an increased parameter reading or a decreased parameter reading from one or more sensors of the plurality of sensors compared to a baseline measurement;
wherein the computing device (400) is capable of deriving a spatiotemporal data set from the plurality of parameter measurements, detecting noise or disturbances in the spatiotemporal data set, and adjusting, based on the plurality of parameter measurements, the baseline measurement with respect to the noise or disturbances.

2. The system (100) of claim 1, wherein one or more sensors of the plurality of sensors comprise a pressure sensor, an electromagnetic sensor, a capacitive sensor, a resistive sensor, or a combination thereof.

3. The system (100) of claim 2, wherein the one or more parameters measured by each sensor (201) of the plurality of sensors is an electrical or analog-to-digital signal selected from a group comprising a capacitance measurement, a radar measurement, an optical measurement, or a combination thereof.

4. The system (100) of claim 3, wherein the computing device (400) is further capable of converting the capacitance measurement into a blood pressure measurement.

5. The system (100) of claim 4 further comprising an attachment component connected to the sensor array (200) for attaching the sensor array (200) to an external surface, wherein the attachment component is selected from a group comprising a strap and an adhesive.

6. The system (100) of claim 5, wherein the external surface is a portion of skin of a patient covering an artery or another layer such as surgical dressing disposed on a portion of skin of the patient.

7. The system (100) of claim 1, wherein the electronic board (300) transmits the plurality of parameter measurements to the computing device (400) through low-energy Bluetooth transmissions.

8. The system (100) of claim 1, wherein the electronic board (300) further comprises an adaptive filter for subtracting noise, creep, hysteresis, motion, or a combination thereof from the plurality of parameter measurements.

9. The system (100) of claim 1, wherein the computing device (400) is further capable of measuring pulse transit time between a first sensor of the plurality of sensors and a second sensor of the plurality of sensors.

10. The system (100) of claim 1, wherein the computing device (400) is further capable of analyzing a pulse wave gathered by one or more sensors of the plurality of sensors.

11. A system (100) for tracking a measured signal and utilizing spatiotemporal data to adjust the measured signal:
a. a sensor array (200) comprising a plurality of sensors, each sensor (201) capable of measuring one or more parameters;
b. an electronic board (300) communicatively coupled to the sensor array (200), the electronic board (300) comprising:
i. a first communication component (301),
ii. a first processor (302) capable of executing computer-readable instructions, and
iii. a first memory component (303) comprising computer-readable instructions, the computer-readable instructions comprising:
A. receiving, from the sensor array (200), a plurality of parameter measurements, and
B. transmitting, by the first communication component (301), the plurality of parameter measurements; and
c. a computing device (400) communicatively coupled to the electronic board (300), the computing device (400) comprising:
i. a second communication component (401),
ii. a second processor (402) capable of executing computer-readable instructions, and
iii. a second memory component (403) comprising computer-readable instructions, the computer-readable instructions comprising:
A. receiving, by the second communication component (401), a first plurality of parameter measurements and a second plurality of parameter measurements from the electronic board (300),
B. establishing, based on the first plurality of parameter measurements, a baseline measurement,
C. deriving a spatiotemporal data set from the second plurality of parameter measurements,
D. detecting noise or disturbances in the spatiotemporal data set, and
E. adjusting, based on the second plurality of parameter measurements, the baseline measurement with respect to the noise or disturbances;
wherein the change to the sensor array (200) is detected by measuring an increased parameter reading or a decreased parameter reading from one or more sensors of the plurality of sensors compared to a baseline measurement.

12. A method for selecting one or more signals of interest, tracking the one or more selected signals, and utilizing spatiotemporal data to adjust the one or more selected signals for noise or disturbances:
a. measuring, by a sensor array (200) comprising a plurality of sensors, a first plurality of parameter measurements,
wherein each sensor (201) of the plurality of sensors is capable of measuring one or more parameters;
b. measuring, by the sensor array (200), a second plurality of parameter measurements;
c. transmitting, by an electronic board (300) communicatively coupled to the sensor array (200), the first plurality of parameter measurements and the second plurality of parameter measurements to a computing device (400) communicatively coupled to the electronic board (300);
d. establishing, by the computing device (400), a baseline measurement based on the first plurality of parameter measurements;
e. deriving a spatiotemporal data set from the second plurality of parameter measurements;
f. detecting noise, disturbances, or one or more signals of interest in the spatiotemporal data set; and
g. adjusting, based on the second plurality of parameter measurements, the baseline measurement with respect to the noise, disturbances, or one or more signals of interest;
wherein the change to the sensor array (200) is detected by measuring an increased parameter reading or a decreased parameter reading from one or more sensors of the plurality of sensors compared to a baseline measurement.

13. The method of claim 12, wherein one or more sensors of the plurality of sensors comprise a pressure sensor, an electromagnetic sensor, a capacitive sensor, a resistive sensor, or a combination thereof.

14. The method of claim 13 further comprising converting, by the computing device (400), the plurality of capacitance measurements into a blood pressure measurement.

15. The method of claim 14 further comprising attaching, by an attachment component connected to the sensor array (200), the sensor array (200) to an external surface.

16. The method of claim 15, wherein the external surface is a portion of skin of a patient covering an artery or another layer such as surgical dressing disposed on a portion of skin of the patient.

17. The method of claim 12, wherein the electronic board (300) transmits the plurality of parameter measurements to the computing device (400) through low-energy Bluetooth transmissions.

18. The method of claim 12 further comprising filtering, by an adaptive filter, noise, creep, hysteresis, motion, or a combination thereof from the plurality of parameter measurements.

19. The method of claim 12 further comprising measuring, by the computing device (400), pulse transit time between a first sensor of the plurality of sensors and a second sensor of the plurality of sensors.

20. The method of claim 12 further comprising analyzing, by the computing device (400), a pulse wave gathered by one or more sensors of the plurality of sensors.
